(19) United States
(12) Patent Application Publication
(10) Pub. No.: US 2019/0202908 A1
(43) Pub. Date: Jul. 4, 2019

(54) BI-SPECIFIC AGENTS

(71) Applicant: President and Fellows of Harvard College, Cambridge, MA (US)

(72) Inventors: Hongyu Zhao, Malden, MA (US); David P. Corey, Newton, MA (US)

(21) Appl. No.: 15/304,390

(22) PCT Filed: Apr. 15, 2015
(86) PCT No.: PCT/US15/25956

§ 371 Date: Oct. 14, 2016

Related U.S. Application Data
(60) Provisional application No. 61/979,720, filed on Apr. 15, 2014.

(51) Int. Cl. C07K 16/28; C07K 14/435

(52) U.S. Cl. CPC C07K 16/28; C07K 14/43504; C07K 2317/569; A61K 38/00; C07K 2317/52; C07K 2317/526; C07K 2319/30

(57) ABSTRACT

Provided herein are novel compositions and methods related to bi-specific agents having high affinity and specificity for a target molecule.

### RELATED APPLICATIONS

This application claims the benefit of priority to Provisional Application No. 61/979,720, filed Apr. 15, 2014, which is hereby incorporated by reference in its entirety.

### BACKGROUND

Properties that must be taken into account when designing pharmaceutical agents include the agent's affinity for its target (in general, the more tightly an agent binds its target, the lower the effective dose), the agent's specificity for its target (an agent that is highly specific for its target will generally produce fewer harmful off-target effects) and the agent's activity (to be effective, an agent must alter the function of the target in the desired manner). Otherwise promising potential small molecule and peptide therapeutics often suffer from low affinity and/or limited specificity, reducing drug effectiveness and resulting in undesired off-target effects. On the other hand, many potential antibody therapeutics have the necessary affinity and specificity for the target, but have low activity and therefore do not appropriately modulate target function.

The challenges associated with pharmaceutical agent design are evidenced by recent attempts to develop a pain therapeutic by targeting the Nav1.7 protein. Pain is carried to the central nervous system by specialized neurons of the dorsal-root and trigeminal ganglia. To generate nerve signals, pain neurons use Nav1.7, a voltage-gated sodium channel that is not used by other neurons or by muscle. Blocking Nav1.7 inhibits pain signals but not touch sensation, and causes no cognitive deficit. Consequently, Nav1.7 is an attractive target for analgesia, and over thirty companies currently have research programs aimed at blocking Nav1.7, either using small molecule inhibitors or by biological therapeutics. However, to date there are no specific Nav1.7 inhibitors in use. In general, current small molecule drugs and peptide toxins that bind to Nav1.7 have low affinity and specificity, often also affecting one of the nine other sodium channel isoforms that are critical for brain and muscle function. On the other hand, antibodies to Nav1.7 often have high affinity and specificity to the Nav1.7 protein, but have low activity.

Thus, there is a need for novel agents that combine the high affinity and specificity of antibody agents with the high activity of small molecule and peptide therapeutics.

### SUMMARY

Provided herein are novel compositions and methods related to bi-specific agents. In certain embodiments, the agents provided herein combine the high affinity and specificity commonly found in antibody therapeutics agents with the high activity characteristic of many small molecule or peptide therapeutic agents.

In certain aspects, provided herein is a bi-specific agent specific for a target protein. In some embodiments, the agent includes a single chain antibody (sdAb) that specifically binds to a first position of the target protein and a target-interacting moiety that interacts with a second position of the target protein. In some embodiments, the target-interacting moiety is a target-specific polypeptide (e.g., a toxin, such as the Ssm6a centipede toxin of SEQ ID NO: 1), a small molecule (e.g., a small molecule linked to the bi-specific agent by an aldehyde tag) or a second sdAb (e.g., a second sdAb that binds to the second position on the target protein). In some embodiments, the target protein is Nav1.7.

In some embodiments, the bi-specific agent comprises an antibody Fc region. In some embodiments, the sdAb is linked to a first Fc domain and the target-interacting moiety is linked to a second Fc domain, wherein the first and second Fc domains interact to form a heterodimeric protein complex. In some embodiments, the Fc domains are human Fc domains. In some embodiments, the Fc domains are human IgG Fc domains (e.g., IgG1 or IgG2 domains). In some embodiments, the Fc domains include modifications that reduce the likelihood of homodimer formation or increase the likelihood of heterodimer formation. For example, in some embodiments, uncharged amino acids are replaced with charged amino acids on the dimerization interface of one or both of the Fc domains (e.g., in the CH3 domain). In some embodiments, an uncharged amino acid is replaced with a positively charged amino acid in one Fc domain and an uncharged amino acid is replaced with a negatively charged amino acid in the other Fc domain. In some embodiments, the first Fc domain comprises a replacement of the amino acid at position 392 with a negative-charged amino acid and the second Fc domain comprises a replacement of Asp 399, Glu356, Asp356 or Glu357 with a positive-charged amino acid. In some embodiments, the second Fc domain comprises a replacement of the amino acid at position 392 with a negative-charged amino acid and the first Fc domain comprises a replacement of Asp 399, Glu356, Asp356 or Glu357 with a positive-charged amino acid.

In some embodiments, the target protein is an ion channel. In some embodiments, the target protein is Nav1.7. In some embodiments, the target-interacting moiety is a peptide toxin comprising an amino acid sequence of SEQ ID NO: 1. In some embodiments, the sdAb binds to an extracellular epitope of Nav1.7 (e.g., an extracellular epitope having a sequence selected from SEQ ID NOs: 8-22).

In certain aspects, provided herein is a method of modulating the activity of a target protein comprising contacting the target protein with a bi-specific agent described herein. In some embodiments, the agent inhibits the activity of the target protein. In some embodiments, the agent increases the activity of the target protein. In some embodiments, the target protein is an ion channel. In some embodiments, the target protein is Nav1.7.

In some aspects, provided herein is a method of treating a disease or disorder in a subject comprising administering to the subject a bi-specific agent described herein. In some embodiments, the disease or disorder is pain, cancer, an inflammatory disease, an autoimmune disease, an allergy, an infection (e.g., a viral or bacterial infection), cardiovascular disease, transplant rejection, graft versus host disease, osteoporosis, a neurological disease and/or macular degeneration.

In some aspects, provided herein is a method of treating pain in a subject comprising administering to the subject a bi-specific agent comprising a single chain antibody (sdAb) that specifically binds to a first position of Nav1.7 and a target-interacting moiety that interacts with a second position of Nav1.7. In some embodiments, the target-interacting moiety is a Nav1.7-specific polypeptide. In some embodiments, the target-specific polypeptide is a toxin. In some embodiments, the target-interacting moiety is a peptide toxin comprising an amino acid sequence of SEQ ID NO: 1. In some embodiments, the sdAb binds to an extracellular epitope of Nav1.7. In some embodiments, the extracellular epitope has a sequence selected from the group consisting of SEQ ID NOs: 8-22. In some embodiments, the bi-specific agent comprises an antibody Fc region. In some embodiments, the sdAb is linked to a first Fc domain and the target-interacting moiety is linked to a second Fc domain, wherein the first and second Fc domains interact to form a heterodimeric protein complex. In some embodiments, the Fc domains are human Fc domains. In some embodiments, the Fc domains are human IgG Fc domains (e.g., IgG1 or IgG2 domains). In some embodiments, the Fc domains include modifications that reduce the likelihood of homodimer formation or increase the likelihood of heterodimer formation. For example, in some embodiments, uncharged amino acids are replaced with charged amino acids on the dimerization interface of one or both of the Fc domains (e.g., in the CH3 domain). In some embodiments, an uncharged amino acid is replaced with a positively charged amino acid in one Fc domain and an uncharged amino acid is replaced with a negatively charged amino acid in the other Fc domain. In some embodiments, the first Fc domain comprises a replacement of the amino acid at position 392 with a negative-charged amino acid and the second Fc domain comprises a replacement of Asp 399, Glu356, Asp356 or Glu357 with a positive-charged amino acid. In some embodiments, the second Fc domain comprises a replacement of the amino acid at position 392 with a negative-charged amino acid and the first Fc domain comprises a replacement of Asp 399, Glu356, Asp356 or Glu357 with a positive-charged amino acid.

In certain aspects, provided herein is a nucleic acid molecule encoding a bi-specific agent described herein or a portion thereof. In some embodiments, the nucleic acid molecule comprises an expression cassette encoding the bi-specific agent described herein. In some embodiments, the nucleic acid molecule comprises a first expression cassette encoding a first polypeptide of a bi-specific agent described herein and a second expression cassette encoding a second polypeptide of a bi-specific agent described herein, wherein the bi-specific agent is a heterodimeric complex of the first and second polypeptides. In some embodiments, the first and second expression cassettes are on separate nucleic acid molecules. In some embodiments, the nucleic acid molecule is a vector. In some embodiments, the nucleic acid molecule is an expression vector.

In certain aspects, provided herein is a cell comprising a nucleic acid molecule described herein. In some embodiments, the cell expresses a bi-specific agent described herein. In some embodiments the cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a human, a monkey, gorilla, chimpanzee). In some embodiments the cell is a cell line. Examples of cell lines include, but are not limited to, P19 cells, HUVAC cells, HEK 293 cells, 283T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

In some aspects, provided herein is a method of producing a cell described herein. In certain embodiments, the method comprises introducing into a cell a nucleic acid molecule described herein. In some embodiments the cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a human, a monkey, gorilla, chimpanzee). In some embodiments the cell is a cell line. Examples of cell lines include, but are not limited to, P19 cells, HUVAC cells, HEK 293 cells, 283T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

In certain aspects, provided herein is a method of generating a bi-specific agent described herein. In certain embodiments, the method includes culturing a cell described herein under conditions such that the cell expresses a bi-specific agent described herein. In some embodiments, the method further comprises isolating the bi-specific agent.

In certain aspects, provided herein is a pharmaceutical composition comprising a bi-specific agent described herein. In some embodiments, the pharmaceutical agent further comprises a pharmaceutically acceptable carrier.

### BRIEF DESCRIPTION OF THE DRAWINGS

FIG. 1 is a schematic diagram of an exemplary bi-specific agent described herein.

FIG. 2 provides amino acid sequences of the Ssm6a centipede toxin, human IgG1 and IgG2 Fc domains, an exemplary linker sequence, an IL2 signal sequence and an Ssm6a-Fc fusion polypeptide.

FIG. 3 provides amino acid sequences of the Nav1.7 protein and exemplary extracellular epitopes of the Nav1.7 protein.

FIG. 4 provides amino acid sequences for a heterodimeric nanobody-Fc fusion protein specific for EGFP.

FIG. 5 provides exemplary nucleic acid and amino acid sequences for Nav1.7-specific nanobodies.

### DETAILED DESCRIPTION

### General

Provided herein are novel compositions and methods related to bi-specific agents having high affinity and specificity for a target molecule. In some embodiments, the bi-specific agents provided herein include a target-interacting moiety that interacts with a first position on a target protein and a single chain antibody (“sdAb”) that binds to a second position on the target protein. In some embodiments, the bi-specific agent has a structure such that the target-interacting moiety is able to interact with the first position on the target protein and the sdAb is able to bind to the second position on the target protein simultaneously. In some embodiments, the target-interacting moiety is an agent that modulates the activity of the target protein (e.g., a small molecule, peptide, toxin or sdAb). In some embodiments, the bi-specific agent comprising the target-interacting moiety has a higher affinity and/or specificity for the target protein than the target-interacting moiety alone.

In some embodiments, provided herein is a bi-specific agent comprising two moieties that each interact with and/or bind to a different position on a target protein, with at least one of the moieties modulating the activity of the target. In some embodiments, the bi-specific nature of the agent causes it to have a high affinity for the target: while one arm may unbind, the other arm will remain bound and allow rapid rebinding of the first arm, so the overall off-rate is very slow. For example, in some embodiments the affinity of the agent for the target is less than 10 nm, 5 nm, 2 nm or 1 nm. The specificity is similarly enhanced by this dual recognition. In some embodiments, each target-interacting and/or target-binding moiety is fused to an Fc domain of a human IgG molecule. Two disulfide bonds dimerize the Fc domains. In some embodiments, hetero-dimerization is promoted by altering the charge on the CH3 region of the IgG domain to create electrostatically polarized Fc domains that preferentially assemble in complementary pairs (e.g., as described in Gunasekaran et al., J. Biol. Chem. 285:19637-19646 (2010) and U.S. Pat. No. 8,592,562, each of which is hereby incorporated by reference). Dimerization of the two Fc domains (forming a heterodimer) results in the formation of the bi-specific agent.

In some embodiments, the agents provided herein are pain inhibitors that function through the inhibition of Nav1.7 activity. For example, in some embodiments, the target-interacting moiety of the bi-specific agents described herein includes the Ssm6a toxin from the Chinese red-headed centipede, which blocks Navl.7 (described in Yang et al., PNAS 110:17534-17539 (2013), incorporated by reference). This centipede toxin is a 46-amino-acid peptide held in a cysteine-knot configuration with high stability by three disulfide bonds. Its affinity for Nav1.7 is about 25 nM, but it has only moderate specificity and cross-reacts with Nav1.2. The Ssm6a toxin has good activity: in animal experiments it blocks pain at doses similar to morphine. Although stable in solution, it is cleared from blood quickly because it is small and therefore has only 4-hour persistence in mouse. In some embodiments, the Ssm6a toxin is fused to the Fc domain of a human IgG. In some embodiments, the bi-specific agent described herein also includes a sdAb (e.g., a variable region of a camelid antibody) specific for Nav1.7 (e.g., an extracellular epitope of Nav1.7), fused to a second Fc domain of a human IgG.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “agent” is used herein to denote a chemical compound, a small molecule, or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject. As used herein, the term “agent” is not limited to therapeutic agents and encompasses agents useful in other applications, including diagnostics, agriculture, research and manufacturing

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

The term “binding” or “interacting” refers to an association, which may be a stable association, between two molecules, e.g., between an agent and a target protein or agent due to, for example, electrostatic, hydrophobic, ionic and/or hydrogen-bond interactions. An agent that interacts with a target protein may modulate the activity of the target protein.

As used herein, the term “bi-specific agent” refers to an agent comprising at least two target-interacting domains, wherein the two target interacting domains are capable of simultaneously interacting with two different positions on a target protein.

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which an antibody is capable of binding.

An “expression vector” is a vector which is capable of promoting expression of a nucleic acid incorporated therein. Typically, the nucleic acid to be expressed is “operably linked” to a transcriptional control element, such as a promoter and/or an enhancer, and is therefore subject to transcription regulatory control by the transcriptional control element.

The term “modulation”, when used in reference to a functional property or biological activity or process (e.g., enzyme activity or receptor binding), refers to the capacity to either up regulate (e.g., activate or stimulate), down regulate (e.g., inhibit or suppress) or otherwise change a quality of such property, activity or process. In certain instances, such regulation may be contingent on the occurrence of a specific event, such as activation of a signal transduction pathway, and/or may be manifest only in particular cell types.

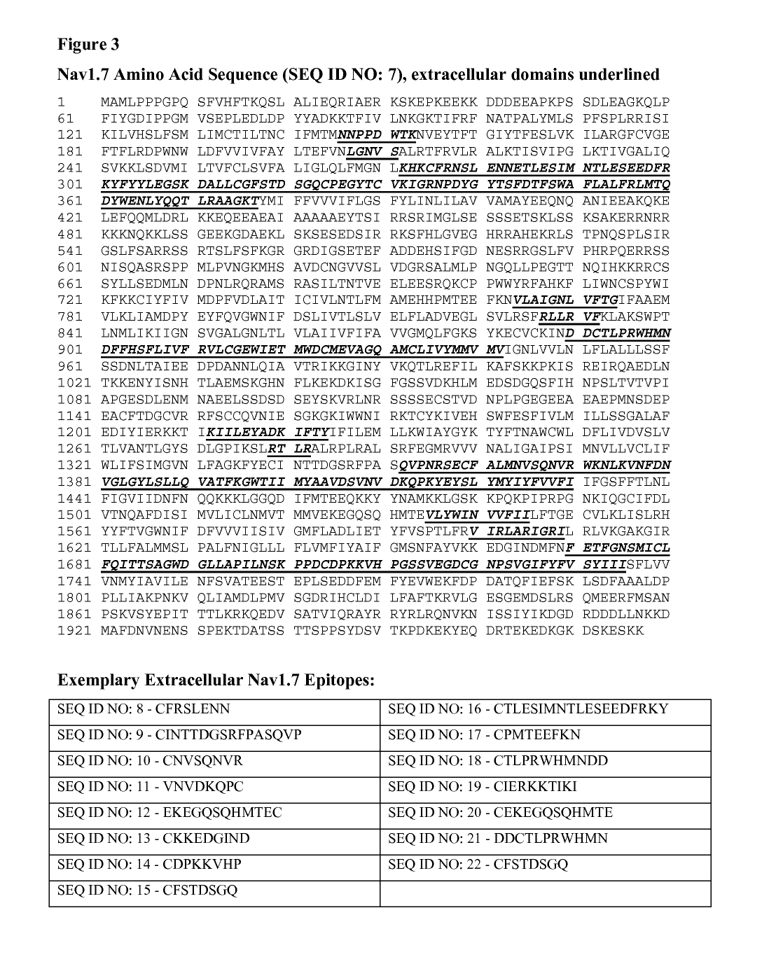

As used herein “Nav 1 .7” refers to the sodium ion channel encoded by the SCN9A gene. The Nav1.7 mRNA sequence is indexed at NCBI accession number NM_002977.3, which is herein incorporated by reference. The Nav1.7 amino acid sequence is indexed at NCBI accession number NP_002968.1, which is herein incorporated by reference, and is provide in FIG. 3 and SEQ ID NO: 7. The extracellular epitopes of Nav1.7 include amino acids 146-153, 206-211, 272-378, 764-774, 827-832, 890-942, 1212-1224, 1279-1282, 1352-1430, 1535-1545, 1600-1609 and 1670-1735 of SEQ ID NO: 2.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

As used herein, the term “single chain antibody,” “single domain antibody” or “sdAb” refers to an antibody fragment having a single monomeric variable antibody domain. Single chain antibodis (also referred to as NANOBODIES®) were initially engineered from heavy-chain antibodies found in camelids. Single chain antibodies and methods for producing single chain antibodies are described in, for example, U.S. Pat. Nos. 5,759,808, 7,943,129, 8,507,748, 8,280,711 and 8,257,705, each of which is incorporated by reference in its entirety.

As used herein, “specific binding” refers to the ability of an antibody (e.g., a single chain antibody) or peptide to bind to an epitope on a target protein. Typically, an antibody or peptide specifically binds to an epitope on a target protein with an affinity corresponding to a K_{D} of about 10⁻⁷ M or less, and binds to the epitope on the target protein with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” refers to a human or a non-human animal, such as a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee).

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Bi-Specific Agents

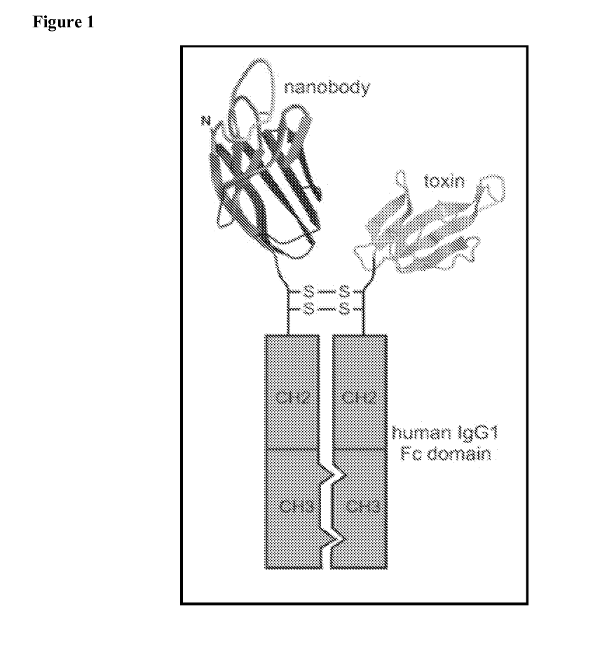

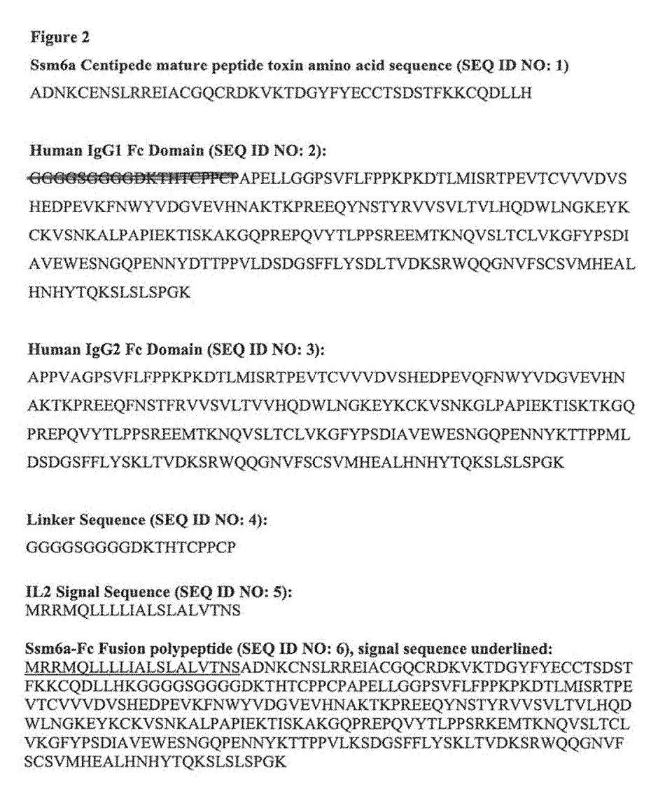

In certain aspects, provided herein is a bi-specific agent specific for a target protein. In some embodiments, the agent includes a target-interacting moiety that interacts with a first position of the target protein and a single chain antibody (sdAb) that specifically binds to a second position of the target protein. In some embodiments, the target-interacting moiety and the sdAb are positioned on the bi-specific agent such that both target-interacting moiety and the sdAb can simultaneously interact with a single target protein. In some embodiments, target-interacting moiety and the sdAb are each fused to an Fc domain of an immunoglobulin molecule and the bi-specific agent comprises a heterodimeric complex of the Fc domain fusion polypeptides. An exemplary embodiment of a bi-specific agent described herein is illustrated in FIG. 1.

In some embodiments, the target-interacting moiety can be any moiety that interacts with and/or binds to a target protein. In some embodiments, the target-interacting moiety can be an agonist or an antagonist of the target protein. In some embodiments, the target-interacting moiety is a sdAb, a polypeptide, a toxin, a cytokine, a chemokine, a growth factor, a drug, an ion channel blocker and/or a small molecule. In some embodiments, the target-interacting moiety is a Ssm6a centipede toxin comprising an amino acid sequence of SEQ ID NO: 1. In some embodiments, the target-interacting moiety is linked to a polypeptide of the bi-specific agent by an aldehyde tag.

In some embodiments, the target-interacting moiety is a sodium channel peptide toxin. In some embodiments, the peptide toxin is selected from the group consisting of μ-Conotoxin BuIIIA, μ-Conotoxin BuIIIB, μ-Conotoxin CnIIIA, μ-Conotoxin CnIIIC, μ-Conotoxin GIIIA, μ-Conotoxin KIIIA, μ-Conotoxin MIIIA, μ-Conotoxin PIIIA, μ-Conotoxin SIIIA, μ-Conotoxin SmIIIA, μ-Conotoxin SxTIIIA, μ-Conotoxin TIIIA and μO§-conotoxin GVIIJ. Such peptide toxins are described, for example, in Wilson et al., Proc. Natl. Acad. Sci. USA 108:10302-10307 (2011), Favreau, Br. J. Pharmacol. 166:1654-1668 (2012), each of which is hereby incorporated by reference in its entirety. In some embodiments, the toxin is β-TRTX-Tp1a, β-TRTX-Tp2a, β-TRTX-Ps1a,β-TRTX-Cm1a or β-TRTX-Cm1b. In some embodiments, the peptide toxin is an artificial peptide based on conotoxins described in Stevens et al., J Biol Chem. 287:31382 (2012) or Brady et al., Mar Drugs. 11:2293-313 (2013), each of which is hereby incorporated by reference in its entirety. In some embodiments, the toxin is Hainantoxin-III, Hainantoxin-IV or Hainantoxin-V. In some embodiments, the toxin is rPnTx1 (described in Silva et al., Biochimie 94:2756-2763 (2012), which is hereby incorporated by reference in its entirety. In some embodiments, the toxin is a synthetic TTX or a derivative of synthetic TTX. In some embodiments, the toxin is a guanidinium based sodium channel blocker. In some embodiments, the toxin is BDS-I, anthopleurin, ATX-1 or ATX2. In some embodiments, the target-interacting moiety is a small molecule sodium channel inhibitor, such as PF-04856264, described in McCormack et al., Proc. Natl. Acad. Sci. USA 110:E2724-32 (2013), which is hereby incorporated by reference in its entirety.

In some embodiments, the target interacting moiety is a neurotoxin. In some embodiments the neurotoxin is selected from the group consisting of Agitoxin, alpha-bungarotoxin, Apamine, Atracotoxin, Batrachotoxin, Botulinum toxin, Charybdotoxin, Chlorotoxin, Cobratoxin, Conotoxin, α-GI, α-GID, ω-MVIIA, ω-CVID, χ-MrIA, ρ-TIA, Conantokin G, Contulakin G, Crotoxin, Dendrotoxin, Erabutoxin, Grammotoxin SIA, GsMTx4, Homobatrachotoxin, HWTX-1(huwentoxin-1), Iberiotoxin, Joro spider toxin, Kaliotoxin, Kurtoxin, Latrotoxin, Margatoxin, Philanthotoxin, Phrixotoxin, SNX-482, Stichodactyla Toxin, Taicatoxin, Texilotoxin, Tityustoxin-K and Versutoxin.

In some embodiments, the bi-specific agent comprises a sdAb that binds to the target molecule (e.g., an extracellular domain of a target molecule). In some embodiments, the sdAb is a humanized sdAb. Methods for the identification and selection of sdAbs against a specific target are well known in the art. For example, sdAbs can be obtained from the immunization of dromedaries, camels, llamas, alpacas or sharks with the antigen of interest followed by the subsequent isolation of the mRNA coding for sdAbs produced. SdAbs specific for a target can also be identified by screening libraries, such as phase-display or yeast-display libraries. In some embodiments, the sdAb binds to Nav1.7 (e.g., SEQ ID NO: 7). In some embodiments, the sdAb binds to an extracellular epitope of Nav1.7 (e.g., an extracellular epitope having a sequence selected from SEQ ID NOs: 8-22). Methods for the production of sdAbs are provided, for example, in U.S. Pat. Nos. 5,759,808, 7,943,129, 8,507,748, 8,280,711 and 8,257,705, each of which is incorporated by reference.

The sdAb can be linked to the target-interacting moiety either directly (e.g., via a polypeptide linker or other chemical linker) or indirectly (e.g., via polypeptides that interact with each other or dimer-forming protein domains). In some embodiments, the bi-specific agent comprises an antibody Fc region. In some embodiments, the sdAb is linked to a first Fc domain and the target-interacting moiety is linked to a second Fc domain, wherein the first and second Fc domains interact to form a heterodimeric protein complex. In some embodiments, the Fc can be of any isotype (e.g., IgA, IgD, IgE, IgG or IgM). In some embodiments, the heavy chain constant region is an IgG constant region (e.g., IgG1, IgG2, IgG3 or IgG4). In some embodiments, the heavy chain constant region can be from any species. For example, in some embodiments, the IgG constant region is a human, mouse, rat, rabbit, donkey, monkey, hamster, sheep, dog or cat constant region. In some embodiments, the Fc domains are human Fc domains. In some embodiments, the Fc domains are human IgG Fc domains (e.g., IgG1 or IgG2 domains).

Mixing equal amounts of BTX-Fc with target-interacting moiety-Fc would lead to formation of homodimers as well as heterodimers. In some embodiments, the heterodimers are purified from the homodimers using standard purification techniques. In some embodiments, the Fc domains include modifications that reduce the likelihood of homodimer formation or increase the likelihood of heterodimer formation. Methods for reducing the likelihood of heterodimer formation are known in the art and described, for example, in U.S. Pat. No. 8,592,562, which is hereby incorporated by reference. For example, in some embodiments, uncharged amino acids are replaced with charged amino acids on the dimerization interface of one or both of the Fc domains (e.g., in the CH3 domain). In some embodiments, an uncharged amino acid is replaced with a positively charged amino acid in one Fc domain and an uncharged amino acid is replaced with a negatively charged amino acid in the other Fc domain. In some embodiments, the first Fc domain comprises a replacement of the amino acid at position 392 with a negative-charged amino acid and the second Fc domain comprises a replacement of Asp 399, Glu356, Asp356 or Glu357 with a positive-charged amino acid. In some embodiments, the second Fc domain comprises a replacement of the amino acid at position 392 with a negative-charged amino acid and the first Fc domain comprises a replacement of Asp 399, Glu356, Asp356 or Glu357 with a positive-charged amino acid. The residue designations are based on the EU numbering scheme of Kabat, which also corresponds to the numbering in the Protein Data Bank (PDB).

In some embodiments, the bi-specific agents provided herein comprise a signal sequence when initially translated. For example, in some embodiments, the bi-specific agents provided herein comprise an IL2 signal sequence of SEQ ID NO: 5.

In some embodiments, the bi-specific agents provided herein are heterodimers of two Fc domain fusion polypeptides. In some embodiments, the structure of the first Fc domain fusion polypeptide has an amino acid sequence represented by the following formula:

[S]-[sdAb]-[L]-[Fc]

Wherein [S] represents an amino acid sequence encoding a signal sequence (e.g., an IL2 signal sequence of SEQ ID NO: 5); [sdAb] represents an amino acid sequence encoding an sdAb specific for the target; [L] represents an amino acid sequence encoding a linker peptide (e.g., a linker polypeptide of SEQ ID NO: 4); and [Fc] represents an amino acid sequence encoding a Fc domain (e.g., an Fc domain of SEQ ID NO: 2 or SEQ ID NO: 3). In some embodiments, the amino acid sequences encoding the signal sequence and/or the linker peptide are optional. In some embodiments, the second Fc domain fusion polypeptide has an amino acid sequence represented by the following formula:

[S]-[IM][L]-[Fc]

Wherein [S] represents an amino acid sequence encoding a signal sequence (e.g., an IL2 signal sequence of SEQ ID NO: 5); [IM] represents an amino acid sequence encoding an interacting moiety specific for the target (e.g., an amino acid sequence encoding the Ssm6a toxin of SEQ ID NO: 1); [L] represents an amino acid sequence encoding a linker peptide (e.g., a linker polypeptide of SEQ ID NO: 4); and [Fc] represents an amino acid sequence encoding a Fc domain (e.g., an Fc domain of SEQ ID NO: 2 or SEQ ID NO: 3).

In some embodiments, the bi-specific agent can be specific for any target protein. In some embodiments, the target protein is an ion channel, a cytokine, a cytokine receptor, a G protein-coupled receptor, a hormone receptor and/or a membrane transport protein. In some embodiments, the target protein is Nav1.7 (e.g., SEQ ID NO:7). In some embodiments, the target protein is Nav1.8. In some embodiments, the target protein is TRPA1.

In some embodiments, the bi-specific agents disclosed herein are able to bind to a target protein with high affinity. In some embodiments, the complex binds to a target protein with an apparent affinity of at least 20 nM, 10 nM, 5 nM, 1 nM, 500 pM, 250 pM, 200 pM, 150 pM, 100 pM, 90 pM, 80 pM or 70 pM. Standard assays to evaluate the binding ability of the complexes are known in the art, including for example, ELISAs, Western blots and RIAs. The binding kinetics (e.g., binding affinity) of the complexes also can be assessed by standard assays known in the art, such as by Biacore analysis.

In some embodiments, bi-specific agents disclosed herein can be produced by recombinant DNA techniques. Alternatively, agents disclosed herein can be chemically synthesized using standard peptide synthesis techniques. In some embodiments, the agents disclosed herein can be purified (e.g., from cells expressing the complexes) by an appropriate purification scheme using standard protein purification techniques. For example, agents comprising an Fc region can be isolated using a Protein A or Protein G column.

In some embodiments, agents disclosed herein comprise an amino acid sequence substantially identical to SEQ ID NO: 1, SEQ ID NO: 2, SEQ ID NO: 3, SEQ ID NO: 4, SEQ ID NO: 5 and/or SEQ ID NO: 6. Accordingly, in another embodiment, the agents comprise an amino acid sequence at least about 80%, 85%, 90%, 91%, 92%, 93%, 94%, 95%, 96%, 97%, 98% or 99% identical to identical SEQ ID NO: 1, SEQ ID NO: 2, SEQ ID NO: 3, SEQ ID NO: 4, SEQ ID NO: 5 and/or SEQ ID NO: 6.

In certain embodiments, the agents disclosed herein comprise an amino acid identical to SEQ ID NO: 1, SEQ ID NO: 2, SEQ ID NO: 3, SEQ ID NO: 4, SEQ ID NO: 5 and/or SEQ ID NO: 6 except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the polypeptides described herein can be replaced with other amino acid residues from the same side chain family and the altered antibody can be tested for retained function using the functional assays described herein.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The agents described herein can be produced in prokaryotic or eukaryotic host cells by expression of polynucleotides encoding a complex of the disclosed herein. Alternatively, such complexes can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N. Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Nucleic Acids

In certain aspects, provided herein are nucleic acid molecules that encode the polypeptides and agents described herein. The nucleic acids may be present, for example, in whole cells, in a cell lysate, or in a partially purified or substantially pure form. Nucleic acids provided herein can be obtained using standard molecular biology techniques. For example, nucleic acid molecules described herein can be cloned using standard PCR techniques or chemically synthesized.

In certain embodiments, provided herein are vectors that contain the nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”).

In certain embodiments, provided herein are cells and non-human organisms that contain a nucleic acid described herein (e.g., a nucleic acid encoding polypeptide or complex described herein). In some embodiments, the cell is a bacterial cell, a yeast cell, a plant cell or an animal cell. In some embodiments, the cell is a mammalian cell, such as a human cell, a primate cell or a rodent cell. In some embodiments, the transgenic non-human organism is a plant, an animal, a yeast or a bacterium. In some embodiments, the transgenic non-human organism is a mammal. In some embodiments, the transgenic non-human organism is a mouse.

In certain embodiments the nucleic acid described herein is operably linked to a transcription control element such as a promoter. In some embodiments the cell transcribes the nucleic acid and thereby expresses a polypeptide or complex described herein. The nucleic acid molecule can be integrated into the genome of the cell or it can be extrachromasomal.

In certain aspects, provided herein is a cell comprising a nucleic acid molecule described herein. In some embodiments, the cell expresses a bi-specific agent described herein. In some embodiments the cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a human, a monkey, gorilla, chimpanzee). In some embodiments the cell is a cell line. Examples of cell lines include, but are not limited to, P19 cells, HUVAC cells, HEK 293 cells, 283T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

In some aspects, provided herein is a method of producing a cell described herein. In certain embodiments, the method comprises introducing into a cell a nucleic acid molecule described herein. In some embodiments the cell is a vertebrate cell, such as a mammalian cell including non-primate cells (e.g., cells from a cow, pig, horse, donkey, goat, camel, cat, dog, guinea pig, rat, mouse, sheep) and primate cells (e.g., a cell from a human, a monkey, gorilla, chimpanzee). In some embodiments the cell is a cell line. Examples of cell lines include, but are not limited to, P19 cells, HUVAC cells, HEK 293 cells, 283T cells, 3T3 cells, 721 cells, 9L cells, A2780 cells, A172 cells, A253 cells, A431 cells, CHO cells, COS-7 cells, HCA2 cells, HeLa cells, Jurkat cells, NIH-3T3 cells and Vero cells.

### Pharmaceutical Compositions

In certain embodiments provided herein is a composition, e.g., a pharmaceutical composition, containing at least one agent described herein (e.g., a bi-specific agent described herein) formulated together with a pharmaceutically acceptable carrier. The pharmaceutical compositions provided herein can be administered in combination therapy, i.e., combined with other agents.

The pharmaceutical compositions provided herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions provided herein suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, agents provided herein, which may be used in a suitable hydrated form, and/or the pharmaceutical compositions of the provided herein, are formulated into pharmaceutically-acceptable dosage forms by conventional methods known to those of skill in the art.

### Therapeutic Methods

In some aspects, provided herein is a method of treating a disease or disorder in a subject comprising administering to the subject a bi-specific agent described herein. In some embodiments, the methods provided herein comprise administering to a subject, (e.g., a subject in need thereof), an effective amount of an agent (e.g., a bi-specific agent described herein) that binds to and modulates the activity of a target protein such that the disease or disorder is treated. The compositions provided herein may be delivered by any suitable route of administration.

In some embodiments, the disease or disorder is pain. In some embodiments, the pain is chronic pain. In some embodiments, the pain is cancer pain, joint pain or back pain. In some embodiments, the pain is caused by an inflammatory or autoimmune condition (e.g., arthritis, multiple sclerosis), a genetic disorder (e.g. amyotrophic lateral sclerosis), cancer, an infectious disease and/or physical trauma (e.g., burns, nerve damage). In some embodiments, the pain is associated with childbirth.

In some embodiments, the disease or disorder is cancer. Cancers that may treated include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the disease or disorder is an age-related disease. Age-related diseases include, but are not limited to, Alzheimer's disease, amniotropic lateral sclerosis, arthritis, atherosclerosis, cachexia, cancer, cardiac hypertrophy, cardiac failure, cardiac hypertrophy, cardiovascular disease, cataracts, colitis, chronic obstructive pulmonary disease, dementia, diabetes mellitus, frailty, heart disease, hepatic steatosis, high blood cholesterol, high blood pressure, Huntington's disease, hyperglycemia, hypertension, infertility, inflammatory bowel disease, insulin resistance disorder, lethargy, metabolic syndrome, muscular dystrophy, multiple sclerosis, neuropathy, nephropathy, obesity, osteoporosis, Parkinson's disease, psoriasis, retinal degeneration, sarcopenia, sleep disorders, sepsis and/or stroke.

In some embodiments, the disease or disorder is an immune disease (e.g., an inflammatory disease, an autoimmune disease and/or an allergy). Such diseases include, for example, asthma, inflammatory disease, skin or organ transplantation, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn's disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In some embodiments, the disease or disorder is a neurodegenerative disease. For example, in some embodiments the neurodegenerative disease is ALS, Huntington's disease, Alzheimer's disease, Parkinson's disease, SMA, PLS, PMA, and/or PBP.

In some embodiments, the disease or disorder is an infection (e.g., a viral or bacterial infection). In some embodiments, the infectious disease is the result of an infection by cytomegalovirus, an enterovirus, Epstein-barr virus, hepatitis B virus, hepatitis C virus, herpes simplex virus, HIV, human herpesvirus 6, influenza A, parvovirus, bartonella, borrelia, chlamydia pneumonia, helicobacter pylori, mycobacterium tuberculosis, streptococcus or toxoplasma gondii.

In some embodiments, the subject is undergoing childbirth and/or is expected to undergo childbirth.

In some embodiments, the agents provided herein can be administered in combination therapy, i.e., combined with other agents. For example, an agent provided herein can be administered as part of a conjunctive therapy in combination with a second agent for the treatment of the disease or disorder.

Conjunctive therapy includes sequential, simultaneous and separate, and/or co-administration of the active compounds in a such a way that the therapeutic effects of the first agent administered have not entirely disappeared when the subsequent agent is administered. In certain embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the compounds provided herein employed in the pharmaceutical composition at levels lower than that required in order to achieve the desired therapeutic effect and gradually increase the dosage until the desired effect is achieved.

### EXAMPLES

### Example 1

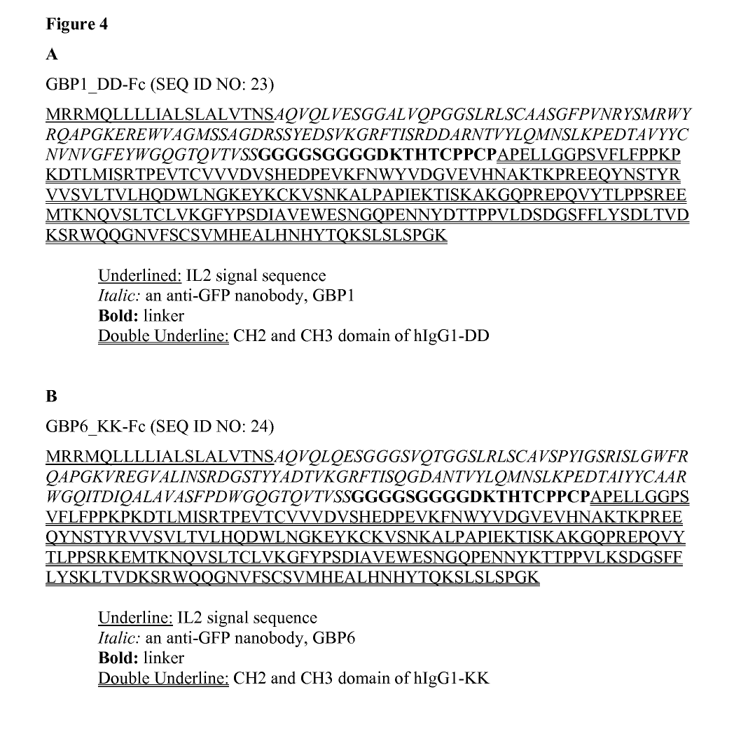

A heterodimeric nanobody-Fc fusion protein specific for EGFP was generated by co-expressing polypeptide GBP1_DD-Fc (sequence provided in FIG. 4A) and polypeptide GBP6_KK-Fc (sequence provided in FIG. 4B) in HEK cells. Using Biacore (SPR), the affinity of the heterodimeric nanobody-Fc fusion protein for EGFP was measured and shown to have a K_{D} of 32.5 pM, as compared to affinities of 101 pM and 2.26 nM for the GPB1 and GPB6 nanobodies alone, respectively.

### Example 2

A homodimeric ssm6a-Fc fusion protein as expressed in HEK cells. The homodimeric fusion protein inhibited Nav1.7 in HEK_Nac1.7 cells with an activity similar to native ssm6a toxin.

### Example 3

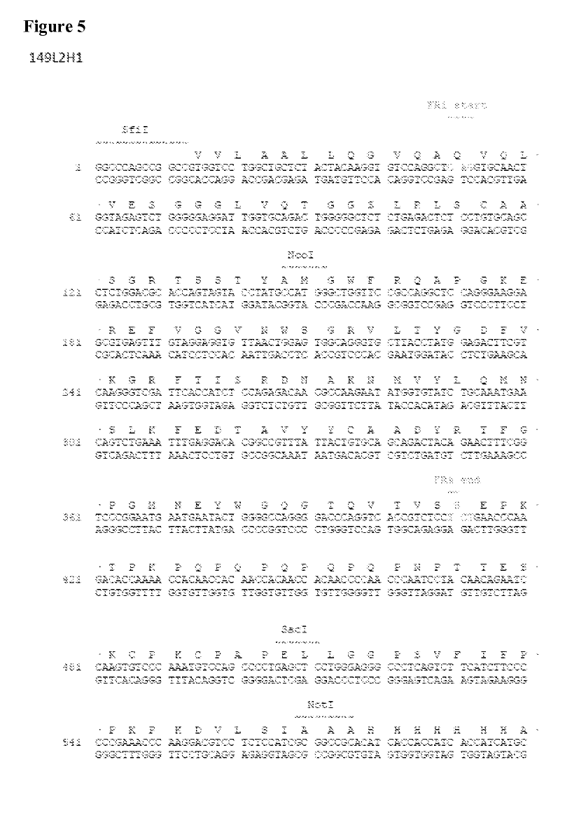

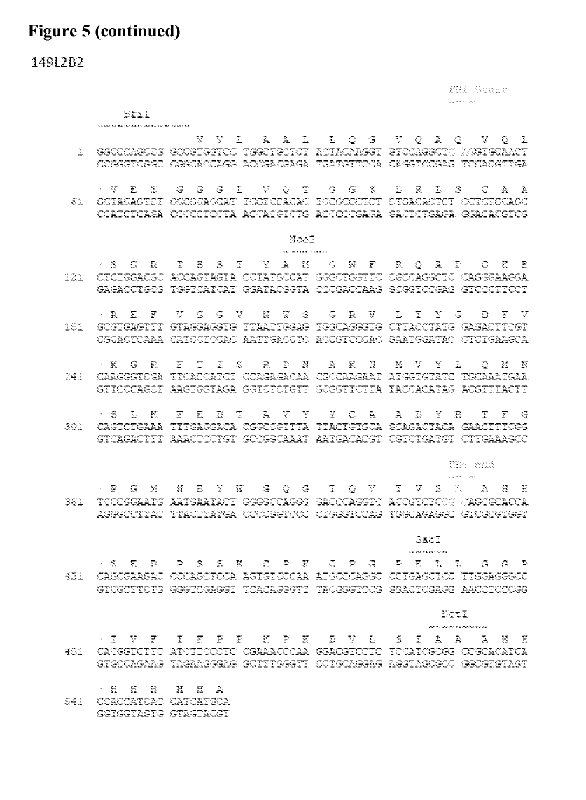

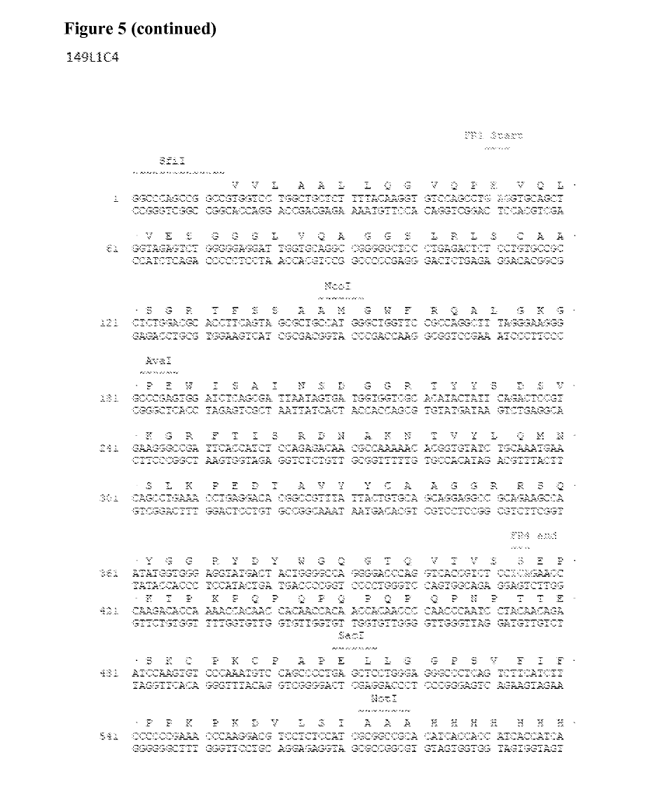

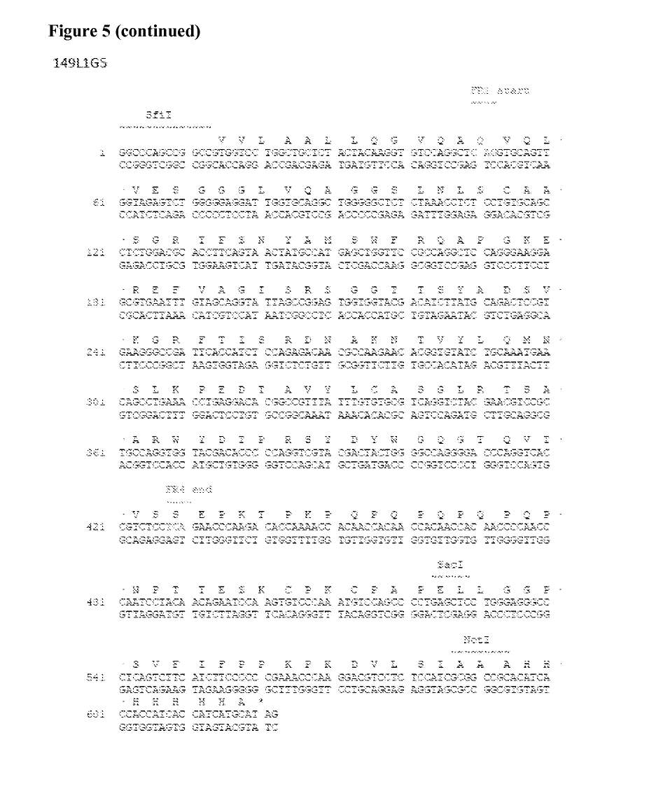

Four nanobodies specific for Nav1.7 were generated that bind to their corresponding epitope with high affinity (0.11 to 1 nM). The nucleic acid and amino acid sequences of the Nav1.7-specific nanobodies (designated 149L2H1, 149L2B2, 149L1C4 and 149L1G5 respectively) are provided in FIG. 5.

All publications, including patents, applications, and GenBank Accession numbers mentioned herein are hereby incorporated by reference in their entirety as if each individual publication or patent was specifically and individually indicated to be incorporated by reference. In case of conflict, the present application, including any definitions herein, will control.

1. A bi-specific agent specific for a target protein, the agent comprising a single chain antibody (sdAb) that specifically binds to a first position of the target protein and a target-interacting moiety that interacts with a second position of the target protein.

2. The bi-specific agent of claim 1, wherein the target-interacting moiety is a target-specific polypeptide.

3. The bi-specific agent of claim 2, wherein the target-specific polypeptide is a toxin.

4. The bi-specific agent of claim 1, wherein the target-interacting moiety is a small molecule.

5. The bi-specific agent of claim 4, wherein the small molecule is linked to the bi-specific agent by an aldehyde tag.

6. The bi-specific agent of claim 1, wherein the target-interacting moiety comprises a second sdAb that specifically binds to the second position of the target protein.

7. The bi-specific agent of claim 1, wherein the sdAb is linked to a first Fc domain and the target-interacting moiety is linked to a second Fc domain, wherein the first and second Fc domains interact to form a heterodimeric protein complex.

8. The bi-specific agent of claim 7, wherein the Fc domains are human Fc domains.

9. The bi-specific agent of claim 8, wherein the Fc domains are human IgG Fc domains.

10. The bi-specific agent of claim 9, wherein the Fc domains comprise modifications that reduce the likelihood of homodimer formation or increase the likelihood of heterodimer formation.

11. The bi-specific agent of claim 10, wherein the modifications are located in in the CH3 domains of the Fc domains.

12. The bi-specific agent of claim 11, wherein the first Fc domain comprises a replacement of the amino acid at position 392 with a negative-charged amino acid and the second Fc domain comprises a replacement of Asp 399, Glu356, Asp356 or Glu357 with a positive-charged amino acid or the second Fc domain comprises a replacement of the amino acid at position 392 with a negative-charged amino acid and the first Fc domain comprises a replacement of Asp 399, Glu356, Asp356 or Glu357 with a positive-charged amino acid.

13. (canceled)

14. The bi-specific agent of claim 1, wherein the target protein is an ion channel.

15. The bi-specific agent of claim 14, wherein the target protein is Nav1.7.

16. The bi-specific agent of claim 15, wherein the target-interacting moiety is a peptide toxin comprising an amino acid sequence of SEQ ID NO: 1.

17. The bi-specific agent of claim 15, wherein the sdAb binds to an extracellular epitope of Nav1.7.

18. The bi-specific agent of claim 17, wherein the extracellular epitope has a sequence selected from the group consisting of SEQ ID NOs: 8-22.

19. The bi-specific agent of claim 16, wherein the sdAb binds to an extracellular epitope of Nav1.7.

20. (canceled)

21. A method of modulating the activity of a target protein comprising contacting the target protein with a bi-specific agent comprising a single chain antibody (sdAb) that specifically binds to a first position of the target protein and a target-interacting moiety that interacts with a second position of the target protein.

22-42. (canceled)

43. A method of treating pain in a subject comprising administering to the subject an agent comprising a single chain antibody (sdAb) that specifically binds to a first position of Nav1.7 and a target-interacting moiety that interacts with a second position of Nav1.7.

44-63. (canceled)


SEQUENCE LISTING (32 sequences)

SEQ ID NO: 1 - protein, 46 aa, Scolopendra mutilans
ADNKCENSLRREIACGQCRDKVKTDGYFYECCTSDSTFKKCQDLLH

SEQ ID NO: 2 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 3 - protein, 216 aa, Homo sapiens
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 19 aa, Artificial Sequence
  Description of Artificial Sequence Synthetic peptide
GGGGSGGGGDKTHTCPPCP

SEQ ID NO: 5 - protein, 20 aa, Unknown
  Description of Unknown IL2 signal sequence
MRRMQLLLLIALSLALVTNS

SEQ ID NO: 6 - protein, 302 aa, Artificial Sequence
  Description of Artificial Sequence Synthetic polypeptide
MRRMQLLLLIALSLALVTNSADNKCNSLRREIACGQCRDKVKTDGYFYECCTSDSTFKKCQDLLHKGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 7 - protein, 1977 aa, Homo sapiens
MAMLPPPGPQSFVHFTKQSLALIEQRIAERKSKEPKEEKKDDDEEAPKPSSDLEAGKQLPFIYGDIPPGMVSEPLEDLDPYYADKKTFIVLNKGKTIFRFNATPALYMLSPFSPLRRISIKILVHSLFSMLIMCTILTNCIFMTMNNPPDWTKNVEYTFTGIYTFESLVKILARGFCVGEFTFLRDPWNWLDFVVIVFAYLTEFVNLGNVSALRTFRVLRALKTISVIPGLKTIVGALIQSVKKLSDVMILTVFCLSVFALIGLQLFMGNLKHKCFRNSLENNETLESIMNTLESEEDFRKYFYYLEGSKDALLCGFSTDSGQCPEGYTCVKIGRNPDYGYTSFDTFSWAFLALFRLMTQDYWENLYQQTLRAAGKTYMIFFVVVIFLGSFYLINLILAVVAMAYEEQNQANIEEAKQKELEFQQMLDRLKKEQEEAEAIAAAAAEYTSIRRSRIMGLSESSSETSKLSSKSAKERRNRRKKKNQKKLSSGEEKGDAEKLSKSESEDSIRRKSFHLGVEGHRRAHEKRLSTPNQSPLSIRGSLFSARRSSRTSLFSFKGRGRDIGSETEFADDEHSIFGDNESRRGSLFVPHRPQERRSSNISQASRSPPMLPVNGKMHSAVDCNGVVSLVDGRSALMLPNGQLLPEGTTNQIHKKRRCSSYLLSEDMLNDPNLRQRAMSRASILTNTVEELEESRQKCPPWWYRFAHKFLIWNCSPYWIKFKKCIYFIVMDPFVDLAITICIVLNTLFMAMEHHPMTEEFKNVLAIGNLVFTGIFAAEMVLKLIAMDPYEYFQVGWNIFDSLIVTLSLVELFLADVEGLSVLRSFRLLRVFKLAKSWPTLNMLIKIIGNSVGALGNLTLVLAIIVFIFAVVGMQLFGKSYKECVCKINDDCTLPRWHMNDFFHSFLIVFRVLCGEWIETMWDCMEVAGQAMCLIVYMMVMVIGNLVVLNLFLALLLSSFSSDNLTAIEEDPDANNLQIAVTRIKKGINYVKQTLREFILKAFSKKPKISREIRQAEDLNTKKENYISNHTLAEMSKGHNFLKEKDKISGFGSSVDKHLMEDSDGQSFIHNPSLTVTVPIAPGESDLENMNAEELSSDSDSEYSKVRLNRSSSSECSTVDNPLPGEGEEAEAEPMNSDEPEACFTDGCVRRFSCCQVNIESGKGKIWWNIRKTCYKIVEHSWFESFIVLMILLSSGALAFEDIYIERKKTIKIILEYADKIFTYIFILEMLLKWIAYGYKTYFTNAWCWLDFLIVDVSLVTLVANTLGYSDLGPIKSLRTLRALRPLRALSRFEGMRVVVNALIGAIPSIMNVLLVCLIFWLIFSIMGVNLFAGKFYECINTTDGSRFPASQVPNRSECFALMNVSQNVRWKNLKVNFDNVGLGYLSLLQVATFKGWTIIMYAAVDSVNVDKQPKYEYSLYMYIYFVVFIIFGSFFTLNLFIGVIIDNFNQQKKKLGGQDIFMTEEQKKYYNAMKKLGSKKPQKPIPRPGNKIQGCIFDLVTNQAFDISIMVLICLNMVTMMVEKEGQSQHMTEVLYWINVVFIILFTGECVLKLISLRHYYFTVGWNIFDFVVVIISIVGMFLADLIETYFVSPTLFRVIRLARIGRILRLVKGAKGIRTLLFALMMSLPALFNIGLLLFLVMFIYAIFGMSNFAYVKKEDGINDMFNFETFGNSMICLFQITTSAGWDGLLAPILNSKPPDCDPKKVHPGSSVEGDCGNPSVGIFYFVSYIIISFLVVVNMYIAVILENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 8 - protein, 8 aa, Unknown
  Description of Unknown extracellular Nav1.7 epitope sequence
CFRSLENN

SEQ ID NO: 9 - protein, 16 aa, Unknown
  Description of Unknown extracellular Nav1.7 epitope sequence
CINTTDGSRFPASQVP

SEQ ID NO: 10 - protein, 8 aa, Unknown
  Description of Unknown extracellular Nav1.7 epitope sequence
CNVSQNVR

SEQ ID NO: 11 - protein, 8 aa, Unknown
  Description of Unknown extracellular Nav1.7 epitope sequence
VNVDKQPC

SEQ ID NO: 12 - protein, 12 aa, Unknown
  Description of Unknown extracellular Nav1.7 epitope sequence
EKEGQSQHMTEC

SEQ ID NO: 13 - protein, 9 aa, Unknown
  Description of Unknown extracellular Nav1.7 epitope sequence
CKKEDGIND

SEQ ID NO: 14 - protein, 8 aa, Unknown
  Description of Unknown extracellular Nav1.7 epitope sequence
CDPKKVHP

SEQ ID NO: 15 - protein, 8 aa, Unknown
  Description of Unknown extracellular Nav1.7 epitope sequence
CFSTDSGQ

SEQ ID NO: 16 - protein, 19 aa, Unknown
  Description of Unknown extracellular Nav1.7 epitope sequence
CTLESIMNTLESEEDFRKY

SEQ ID NO: 17 - protein, 9 aa, Unknown
  Description of Unknown extracellular Nav1.7 epitope sequence
CPMTEEFKN

SEQ ID NO: 18 - protein, 11 aa, Unknown
  Description of Unknown extracellular Nav1.7 epitope sequence
CTLPRWHMNDD

SEQ ID NO: 19 - protein, 10 aa, Unknown
  Description of Unknown extracellular Nav1.7 epitope sequence
CIERKKTIKI

SEQ ID NO: 20 - protein, 12 aa, Unknown
  Description of Unknown extracellular Nav1.7 epitope sequence
CEKEGQSQHMTE

SEQ ID NO: 21 - protein, 11 aa, Unknown
  Description of Unknown extracellular Nav1.7 epitope sequence
DDCTLPRWHMN

SEQ ID NO: 22 - protein, 8 aa, Unknown
  Description of Unknown extracellular Nav1.7 epitope sequence
CFSTDSGQ

SEQ ID NO: 23 - protein, 372 aa, Artificial Sequence
  Description of Artificial Sequence Synthetic polypeptide
MRRMQLLLLIALSLALVTNSAQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYDTTPPVLDSDGSFFLYSDLTVDKSRWQQGNVFSCSVMHEALHNHYTQKSLSLSPGK

SEQ ID NO: 24 - protein, 383 aa, Artificial Sequence
  Description of Artificial Sequence Synthetic polypeptide
MRRMQLLLLIALSLALVTNSAQVQLQESGGGSVQTGGSLRLSCAVSPYIGSRISLGWFRQAPGKVREGVALINSRDGSTYYADTVKGRFTISQGDANTVYLQMNSLKPEDTAIYYCAARWGQITDIQALAVASFPDWGQGTQVTVSSGGGGSGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRKEMTKNQVSLTCLVKGFYPSDIAVEWESNGQPENNYKTTPPVLKSDGSFFLYSKLTVDKSRWQQGNVFSCSVMHEALHNHYTQKSLSLSPGK

SEQ ID NO: 25 - protein, 196 aa, Artificial Sequence
  Description of Artificial Sequence Synthetic polypeptide
VVLAALLQGVQAQVQLVESGGGLVQTGGSLRLSCAASGRTSSTYAMGWFRQAPGKEREFVGGVNWSGRVLTYGDFVKGRFTISRDNAKNMVYLQMNSLKFEDTAVYYCAADYRTFGPGMNEYWGQGTQVTVSSEPKTPKPQPQPQPQPQPNPTTESKCPKCPAPELLGGPSVFIFPPKPKDVLSIAAAHHHHHHHA

SEQ ID NO: 26 - DNA, 600 bp, Artificial Sequence
  Description of Artificial Sequence Synthetic polynucleotide
ggcccagccggccgtggtcctggctgctctactacaaggtgtccaggctcaggtgcaactggtagagtctgggggaggattggtgcagactgggggctctctgagactctcctgtgcagcctctggacgcaccagtagtacctatgccatgggctggttccgccaggctccagggaaggagcgtgagtttgtaggaggtgttaactggagtggcagggtgcttacctatggagacttcgtcaagggtcgattcaccatctccagagacaacgccaagaatatggtgtatctgcaaatgaacagtctgaaatttgaggacacggccgtttattactgtgcagcagactacagaactttcggtcccggaatgaatgaatactggggccaggggacccaggtcaccgtctcctctgaacccaagacaccaaaaccacaaccacaaccacaaccacaaccccaacccaatcctacaacagaatccaagtgtcccaaatgtccagcccctgagctcctgggagggccctcagtcttcatcttccccccgaaacccaaggacgtcctctccatcgcggccgcacatcaccaccatcaccatcatgc

SEQ ID NO: 27 - protein, 182 aa, Artificial Sequence
  Description of Artificial Sequence Synthetic polypeptide
VVLAALLQGVQAQVQLVESGGGLVQTGGSLRLSCAASGRTSSTYAMGWFRQAPGKEREFVGGVNWSGRVLTYGDFVKGRFTISRDNAKNMVYLQMNSLKFEDTAVYYCAADYRTFGPGMNEYWGQGTQVTVSAAHHSEDPSSKCPKCPGPELLGGPTVFIFPPKPKDVLSIAAAHHHHHHHA

SEQ ID NO: 28 - DNA, 559 bp, Artificial Sequence
  Description of Artificial Sequence Synthetic polynucleotide
ggcccagccggccgtggtcctggctgctctactacaaggtgtccaggctcaggtgcaactggtagagtctgggggaggattggtgcagactgggggctctctgagactctcctgtgcagcctctggacgcaccagtagtacctatgccatgggctggttccgccaggctccagggaaggagcgtgagtttgtaggaggtgttaactggagtggcagggtgcttacctatggagacttcgtcaagggtcgattcaccatctccagagacaacgccaagaatatggtgtatctgcaaatgaacagtctgaaatttgaggacacggccgtttattactgtgcagcagactacagaactttcggtcccggaatgaatgaatactggggccaggggacccaggtcaccgtctccgcagcgcaccacagcgaagaccccagctccaagtgtcccaaatgcccaggccctgagctccttggagggcccacggtcttcatcttccctccgaaacccaaggacgtcctctccatcgcggccgcacatcaccaccatcaccatcatgca

SEQ ID NO: 29 - protein, 196 aa, Artificial Sequence
  Description of Artificial Sequence Synthetic polypeptide
VVLAALLQGVQPEVQLVESGGGLVQAGGSLRLSCAASGRTFSSAAMGWFRQALGKGPEWISAINSDGGRTYYSDSVKGRFTISRDNAKNTVYLQMNSLKPEDTAVYYCAAGGRRSQYGGRYDYWGQGTQVTVSSEPKTPKPQPQPQPQPQPNPTTESKCPKCPAPELLGGPSVFIFPPKPKDVLSIAAAHHHHHHH

SEQ ID NO: 30 - DNA, 600 bp, Artificial Sequence
  Description of Artificial Sequence Synthetic polynucleotide
ggcccagccggccgtggtcctggctgctcttttacaaggtgtccagcctgaggtgcagctggtagagtctgggggaggattggtgcaggccgggggctccctgagactctcctgtgccgcctctggacgcaccttcagtagcgctgccatgggctggttccgccaggctttagggaaggggcccgagtggatctcagcgattaatagtgatggtggtcgcacatactattcagactccgtgaagggccgattcaccatctccagagacaacgccaaaaacacggtgtatctgcaaatgaacagcctgaaacctgaggacacggccgtttattactgtgcagcaggaggccgcagaagccaatatggtgggaggtatgactactggggccaggggacccaggtcaccgtctcctcagaacccaagacaccaaaaccacaaccacaaccacaaccacaaccccaacccaatcctacaacagaatccaagtgtcccaaatgtccagcccctgagctcctgggagggccctcagtcttcatcttccccccgaaacccaaggacgtcctctccatcgcggccgcacatcaccaccatcaccatca

SEQ ID NO: 31 - protein, 202 aa, Artificial Sequence
  Description of Artificial Sequence Synthetic polypeptide
VVLAALLQGVQAQVQLVESGGGLVQAGGSLNLSCAASGRTFSNYAMSWFRQAPGKEREFVAGISRSGGTTSYADSVKGRFTISRDNAKNTVYLQMNSLKPEDTAVYLCASGLRTSAARWYDTPRSYDYWGQGTQVTVSSEPKTPKPQPQPQPQPQPNPTTESKCPKCPAPELLGGPSVFIFPPKPKDVLSIAAAHHHHHHHA

SEQ ID NO: 32 - DNA, 622 bp, Artificial Sequence
  Description of Artificial Sequence Synthetic polynucleotide
ggcccagccggccgtggtcctggctgctctactacaaggtgtccaggctcaggtgcagttggtagagtctgggggaggattggtgcaggctgggggctctctaaacctctcctgtgcagcctctggacgcaccttcagtaactatgccatgagctggttccgccaggctccagggaaggagcgtgaatttgtagcaggtattagccggagtggtggtacgacatcttatgcagactccgtgaagggccgattcaccatctccagagacaacgccaagaacacggtgtatctgcaaatgaacagcctgaaacctgaggacacggccgtttatttgtgtgcgtcaggtctacgaacgtccgctgccaggtggtacgacacccccaggtcgtacgactactggggccaggggacccaggtcaccgtctcctcagaacccaagacaccaaaaccacaaccacaaccacaaccacaaccccaacccaatcctacaacagaatccaagtgtcccaaatgtccagcccctgagctcctgggagggccctcagtcttcatcttccccccgaaacccaaggacgtcctctccatcgcggccgcacatcaccaccatcaccatcatgcatag